(19) United States
(12) Patent Application Publication
(10) Pub. No.: US 2022/0218653 A1
(43) Pub. Date: Jul. 14, 2022

(54) CARTRIDGE FOR VAPOR-PHASE CANNABINOID REACTIONS WITHIN A DEVICE

(71) Applicant: Canopy Growth Corporation, Smiths Falls (CA)

(72) Inventors: Christopher ADAIR, Smiths Falls (CA); Ben GEILING, Smiths Falls (CA)

(21) Appl. No.: 17/596,345

(22) PCT Filed: Jun. 11, 2020
(86) PCT No.: PCT/CA2020/050802

§ 371 Date: Dec. 8, 2021

Related U.S. Application Data
(60) Provisional application No. 62/860,169, filed on Jun. 11, 2019.

(51) Int. Cl. A61K 31/352; A61M 11/04; A61K 47/32; A61K 47/34; A61K 47/02; A24F 40/42; A24B 15/167

(52) U.S. Cl. CPC A61K 31/352; A61M 11/041; A61K 47/32; A24B 15/167; A61K 47/02; A24F 40/42; A61K 47/34

(57) ABSTRACT

Disclosed herein is a cartridge for a vape device. The cartridge comprises a housing defining an inlet, an outlet, and an interior chamber that is position between the inlet and the outlet. The inlet, the outlet, and the interior chamber are fluidly connected by a flow path, and the inlet is configured to receive a first cannabinoid. The cartridge also comprises a Lewis-acidic heterogeneous reagent positioned in the interior chamber such that when the flow path passes through the interior chamber, at least a portion of the flow path contacts the Lewis-acidic heterogeneous reagent. The Lewis-acidic heterogeneous reagent has an acidity metric that surpasses a threshold acidity metric for the first cannabinoid such that contact between the Lewis-acidic heterogeneous reagent and the first cannabinoid under reaction conditions defined by a contact temperature and a contact time converts at least a portion of the first cannabinoid into a second cannabinoid.

### CROSS-REFERENCE TO RELATED APPLICATION

This application claims priority to and benefit of U.S. Provisional Patent Application Ser. No. 62/860,169 filed on Jun. 11, 2019, which is hereby incorporated by reference.

### TECHNICAL FIELD

The present disclosure generally relates to vape-device componentry. In particular, the present disclosure relates to cartridges configured for vapor-phase cannabinoid reactions within a vape device.

### BACKGROUND

Vape devices—also referred to as “vaporizers”, “vapes”, “vape pens”, “e-vapes”, “e-cigarettes”, and the like—typically employ a heating element that is configured to volatilize a payload. In this context, volatilization may comprise: (i) heating a solid to induce decomposition, melting, and/or sublimation; (ii) heating a liquid to induce decomposition and/or vaporization; and/or (iii) nebulizing a liquid by expansion through a nozzle. Such processes provide a vapor stream that is inhaled by a user.

Some vape devices are configured for cannabinoid-related applications. In some such instances, vapor-phase compositions that feature a single cannabinoid may be desirable—in other such instances vapor-phase compositions that feature mixtures of cannabinoids may be preferable. Either way, current vape devices are generally not configured to alter the compounds in a cannabinoid-containing vapor towards a particular composition. In other words, known vape devices are limited in that in they lack suitable componentry to modulate the cannabinoid composition of a volatilized payload.

### SUMMARY

The present disclosure acknowledges the foregoing limitations of current vape devices and recognizes the unmet need for vape devices that are configured to modulate the cannabinoid composition of a volatilized payload. Such cartridges may be employed in both recreational and medicinal contexts. The present disclosure advances the art, for example, with the provision of vape-device cartridges that are configured to effect cannabinoid reactions in the vapor phase. As such, the present disclosure provides means to decouple the composition of a cannabinoid-containing vapor-stream from the payload from which it originated. Importantly, the cartridges of the present disclosure utilize a Lewis-acidic heterogeneous reagent to induce such vapor-phase reactions.

In an embodiment, the present disclosure relates to a cartridge for a vape device, the cartridge comprising: a housing defining an inlet, an outlet, and an interior chamber that is positioned between the inlet and the outlet, wherein the inlet, the outlet, and the interior chamber are fluidly connected by a flow path, and wherein the inlet is configured to receive a first cannabinoid; and a Lewis-acidic heterogeneous reagent that is positioned in the interior chamber such that when the flow path passes through the interior chamber, at least a portion of the flow path contacts the Lewis-acidic heterogeneous reagent, wherein the first cannabinoid is volatilized and the Lewis-acidic heterogeneous reagent has an acidity metric that surpasses a threshold acidity metric for the first cannabinoid such that contact between the Lewis-acidic heterogeneous reagent and the first cannabinoid under reaction conditions defined by a contact temperature and a contact time converts at least a portion of the first cannabinoid into a second cannabinoid.

In select embodiments, the present disclosure relates to a cartridge for a vape device, the cartridge comprising: a housing defining a payload reservoir and an outlet; and an atomizer that is in fluid communication with the payload reservoir and the outlet, wherein the atomizer is configured to vaporize at least a portion of a first cannabinoid that is positioned in the payload reservoir, and wherein the atomizer comprises a Lewis-acidic heterogeneous reagent has an acidity metric that surpasses a threshold acidity metric for the first cannabinoid such that contact between the Lewis-acidic heterogeneous reagent and the first cannabinoid under reaction conditions defined by a contact temperature and a contact time converts at least a portion of the first cannabinoid into a second cannabinoid.

In select embodiments, the present disclosure relates to a vape device comprising a cartridge as defined herein.

### BRIEF DESCRIPTION OF THE DRAWINGS

These and other features of the present disclosure will become more apparent in the following description in which reference is made to the appended drawings. The appended drawings illustrate one or more embodiments of the present disclosure by way of example only and are not to be construed as limiting the scope of the present disclosure.

FIG. 1 shows a schematic representation of a cartridge in accordance with a first embodiment of the present disclosure.

FIG. 2 shows a schematic representation of a cartridge in accordance with a second embodiment of the present disclosure.

FIG. 3 shows a high-performance liquid chromatogram for EXAMPLE 1.

FIG. 4 shows a high-performance liquid chromatogram for COMPARISON EXAMPLE 1.

### DETAILED DESCRIPTION

As noted above, the present disclosure recognizes that current vape devices are generally not configured to alter the compounds in a cannabinoid-containing vapor towards a particular composition (i.e. known vape devices are limited in that in they lack suitable componentry to modulate the cannabinoid composition of a volatilized payload). The present disclosure notes that overcoming this shortcoming may advance a plurality of applications in both medicinal and recreational contexts. Decoupling the composition of a cannabinoid-containing vapor-stream from the payload from which it originated may enable the use of new payload compositions, and/or it may provide access to new vapor-phase compositions on the device-scale.

Importantly, the cartridges of the present disclosure utilize Lewis-acidic heterogeneous reagents that are configured to induce vapor-phase cannabinoid reactions on the device scale. In the context of the present disclosure, a “vapor-phase” reaction “on the device scale” is one in which a starting material is converted into a product along a flow path through a device under temperature/pressure/time conditions that are achievable within the device. For example, pressure conditions may be characterized by a modest pressure differential between an inlet and an outlet of the device due to suction created from inhalation by a user.

In the context of the present disclosure, a “cannabinoid reaction” is one in which a first cannabinoid is converted to a second cannabinoid that has a different chemical structure than the first cannabinoid. The first cannabinoid and the second cannabinoid may be isomers in that they may have the same atomic composition.

As used herein, the term “cannabinoid” refers to: (i) a chemical compound belonging to a class of secondary compounds commonly found in plants of genus cannabis, (ii) synthetic cannabinoids and any enantiomers thereof; and/or (iii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In select embodiments of the present disclosure, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11 nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

Within the context of this disclosure, where reference is made to a particular cannabinoid without specifying if it is acidic or neutral, each of the acid and/or decarboxylated forms are contemplated as both single molecules and mixtures.

As used herein, the term “THC” refers to tetrahydrocannabinol. “THC” is used interchangeably herein with “Δ9-THC”.

In select embodiments of the present disclosure, a “first cannabinoid” and/or a “second cannabinoid” may comprise THC (Δ9-THC), Δ8-THC, trans-Δ10-THC, cis-Δ10-THC, THCV, Δ8-THCV, Δ9-THCV, CBD, CBDA, CBDV, CBDVA, CBC, CBCA, CBCV, CBG, CBGV, CBN, CBNV, CBND, CBNDV, CBE, CBEV, CBL, CBLV, CBT, or cannabicitran.

Structural formulae of cannabinoids of the present disclosure may include the following:

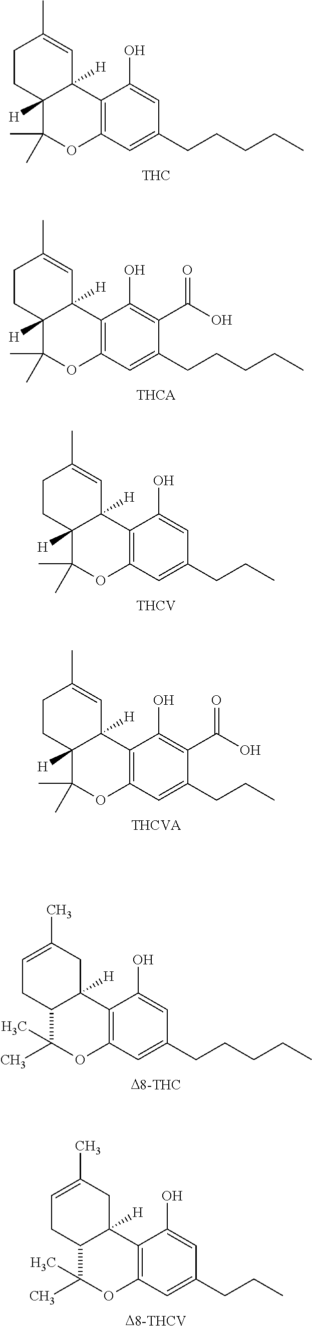

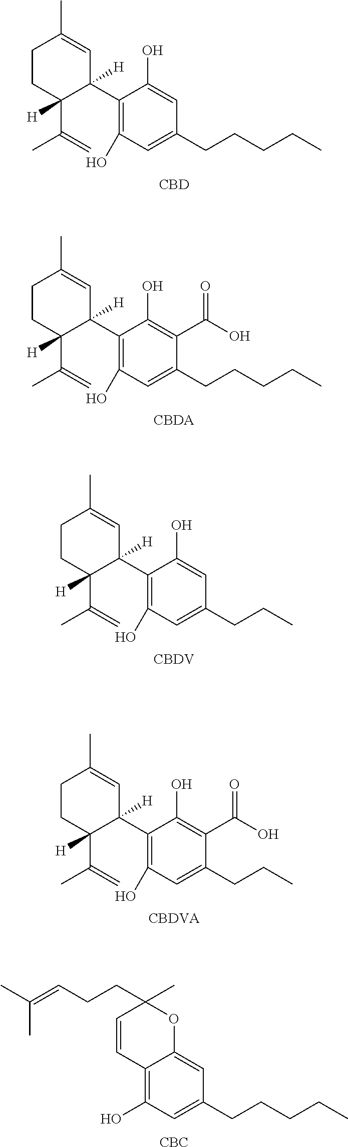

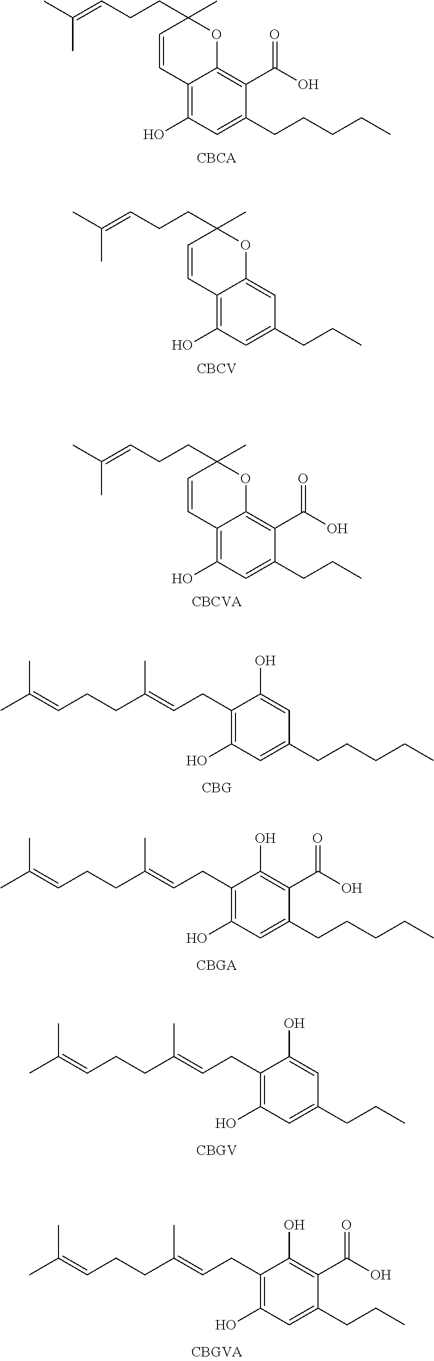

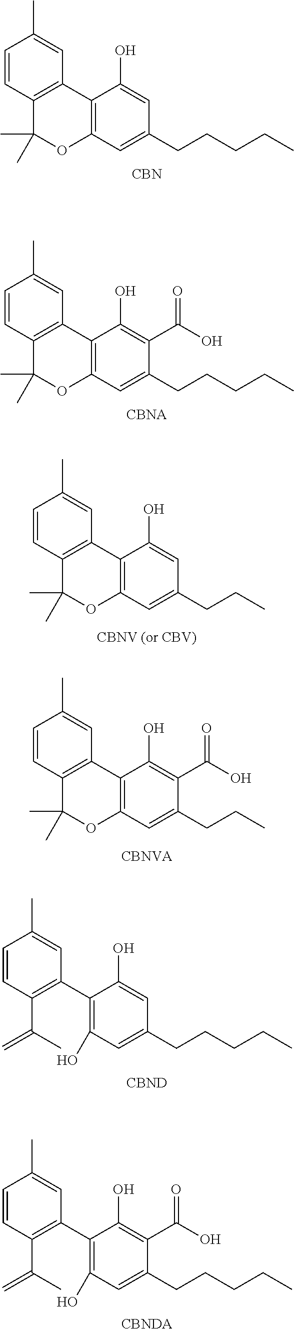

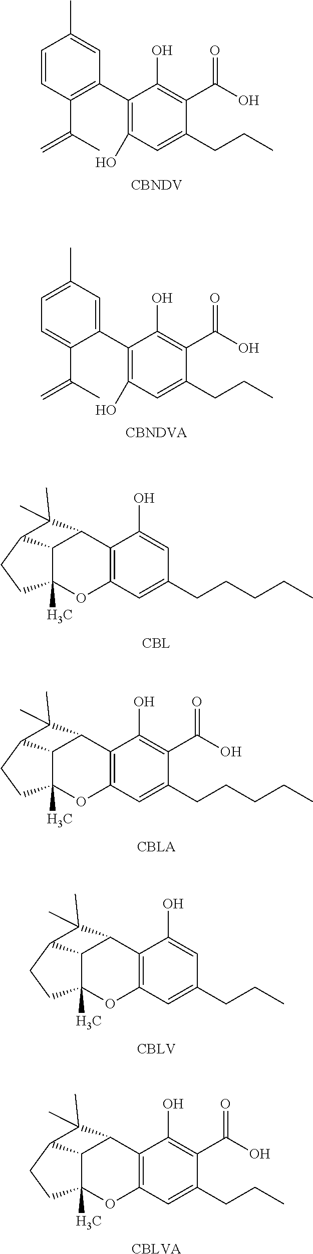

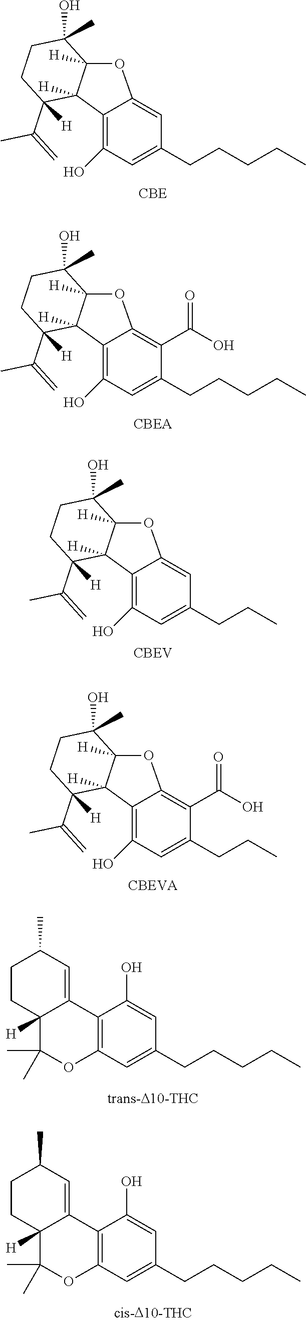

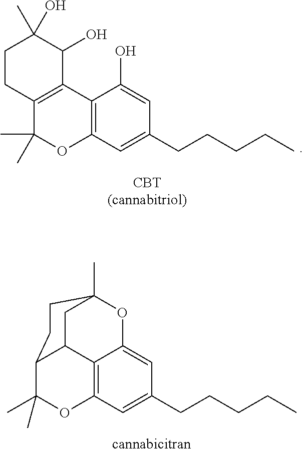

In select embodiments, the first cannabinoid or the second cannabinoid may comprise CBD, CBDV, CBC, CBCV, CBG, CBGV, THC, THCV, or a regioisomer thereof. As used herein, the term “regioisomers” refers to compounds that differ only in the location of a particular functional group.

In select embodiments, the first cannabinoid may be cannabidiol (CBD) and the second cannabinoid may be Δ⁸-tetrahydrocannabinol (Δ⁸-THC) and/or Δ⁹-tetrahydrocannabinol (Δ⁹-THC).

In select embodiments of the present disclosure, the first cannabinoid is Δ⁹-THC or Δ¹⁰-THC.

In select embodiments, the first cannabinoid is a component of a distillate, an isolate, a concentrate, an extract, or a combination thereof.

In an embodiment, the present disclosure relates to a cartridge for a vape device, the cartridge comprising: a housing defining an inlet, an outlet, and an interior chamber that is position between the inlet and the outlet, wherein the inlet, the outlet, and the interior chamber are fluidly connected by a flow path, and the inlet is configured to receive a first cannabinoid; and a Lewis-acidic heterogeneous reagent that is positioned in the interior chamber such that when the flow path passes through the interior chamber, at least a portion of the flow path contacts the Lewis-acidic heterogeneous reagent, wherein the first cannabinoid is volatilized and the Lewis-acidic heterogeneous reagent has an acidity metric that surpasses a threshold acidity metric for the first cannabinoid such that contact between the Lewis-acidic heterogeneous reagent and the first cannabinoid under reaction conditions defined by a contact temperature and a contact time converts at least a portion of the first cannabinoid into a second cannabinoid.

In the context of the present disclosure, the term “contact” and its derivatives is intended to refer to bringing the first cannabinoid and the Lewis-acidic heterogeneous reagent as disclosed herein into proximity such that a chemical reaction can occur. In some embodiments of the present disclosure, the contacting may be by passing at least a portion of the flow path through Lewis-acidic heterogeneous reagent. In some embodiments, the contacting may be by passing at least a portion of the flow path over the surface of a Lewis-acidic heterogeneous reagent.

In select embodiments, the present disclosure relates to a cartridge for a vape device, the cartridge comprising: a housing defining a payload reservoir and an outlet; and an atomizer that is in fluid communication with the payload reservoir and the outlet, wherein the atomizer configured to vaporize at least a portion of a first cannabinoid that is positioned in the payload reservoir, and wherein the atomizer comprises a Lewis-acidic heterogeneous reagent has an acidity metric that surpasses a threshold acidity metric for the first cannabinoid such that contact between the Lewis-acidic heterogeneous reagent and the first cannabinoid under reaction conditions defined by a contact temperature and a contact time converts at least a portion of the first cannabinoid into a second cannabinoid.

In the context of the present disclosure, the relative quantities of a first cannabinoid and a second cannabinoid in a particular composition may be expressed as a ratio—second cannabinoid:first cannabinoid. In select embodiments of the present disclosure, a first cannabinoid may be converted into a mixture of cannabinoid products referred to herein as a second cannabinoid, a third cannabinoid, and so on. The relative quantities of cannabinoid products in a mixture may be referred to with analogous ratios (e.g. second cannabinoid:third cannabinoid). Those skilled in the art will recognize that a variety of analytical methods may be used to determine such ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. In select embodiments of the present disclosure, the compositions provided by the methods of the present disclosure have second cannabinoid:first cannabinoid ratios of greater than 1.0:1.0, meaning the quantity of the second cannabinoid in the composition is greater than the quantity of the first cannabinoid in the composition. For example, the compositions provided by the methods of the present disclosure may have second cannabinoid:first cannabinoid ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; and (viii) greater than about 100.0:1.0. In select embodiments of the present disclosure, the compositions provided by the methods of the present disclosure have second cannabinoid:third cannabinoid ratios of greater than 1.0:1.0, meaning the quantity of the second cannabinoid in the composition is greater than the quantity of the third cannabinoid in the composition. For example, the compositions provided by the methods of the present disclosure may have second cannabinoid:third cannabinoid ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; and (viii) greater than about 100.0:1.0.

In the context of the present disclosure, a Lewis-acidic heterogeneous reagent is one which: (i) comprises one or more sites that are capable of accepting an electron pair from an electron pair donor; and (ii) is substantially not mono-phasic with the reagent. Likewise, in the context of the present disclosure, a Brønsted-acid heterogeneous reagent is one which: (i) comprises one or more sites that are capable of donating a proton to a proton-acceptor; and (ii) is substantially not mono-phasic with the starting material and/or provides an interface where one or more chemical reaction takes place. Importantly, the term “reagent” is used in the present disclosure to encompass both reactant-type reactivity (i.e. wherein the reagent is at least partly consumed as reactant is converted to product) and catalyst-type reactivity (i.e. wherein the reagent is not substantially consumed as reactant is converted to product).

In the context of the present disclosure, the acidity of a Lewis-acidic heterogeneous reagent and/or a Brønsted-acid heterogeneous reagent may be characterized by a variety of parameters, non-limiting examples of which are summarized in the following paragraphs.

As will be appreciated by those skilled in the art who have benefitted from the teachings of the present disclosure, determining the acidity of heterogeneous solid acids may be significantly more challenging than measuring the acidity of homogenous acids due to the complex molecular structure of heterogeneous solid acids. The Hammett acidity function (H₀) has been applied over the last 60 years to characterize the acidity of solid acids in non-aqueous solutions. This method utilizes organic indicator bases, known as Hammett indicators, which coordinate to the accessible acidic sites of the solid acid upon protonation. Typically, a color change is observed during titration with an additional organic base (e.g. n-butylamine), which is measured by UV-visible spectroscopy to quantify acidity. Multiple Hammett indicators with pKa values ranging from +6.8 (e.g. neutral red) to −8.2 (e.g. anthraquinone) are tested with a given solid acid to determine the quantity and strength of acidic sites, which is typically expressed in mmol per gram of solid acid for each indicator. Hammett acidity values may not provide a complete characterization of acidity. For example, accurate measurement of acidity may rely on the ability of the Hammett indicator to access the interior acidic sites within the solid acid. Some solid acids may have pore sizes that permit the passage of small molecules but prevent larger molecules from accessing the interior of the acid. H-ZSM-5 may be a representative example, wherein larger Hammett indicators such as anthraquinone may not be able to access interior acidic sites, which may lead to an incomplete measure of its total acidity.

Temperature-Programmed Desorption (TPD) is an alternate technique for characterizing the acidity of heterogeneous solid acids. This technique typically utilizes an organic base with small molecular size (e.g. ammonia, pyridine, n-propylamine), which may react with the acid sites on the exterior and interior of the solid acid in a closed system. After the solid acid is substantially saturated with organic base, the temperature is increased and the change in organic base concentration is monitored gravimetrically, volumetrically, by gas chromatography, or by mass spectrometry. The amount of organic base desorbing from the solid acid above some characteristic temperature may be interpreted as the acid-site concentration. TPD is often considered more representative of total acidity for solid acids compared to the Hammett acidity function, because the selected organic base is small enough to bind to acidic sites on the interior of the solid acid.

In select embodiments of the present disclosure, TPD values are reported with respect to ammonia. Those skilled in the art who have benefited from the teachings of the present disclosure will appreciate that ammonia may have the potential disadvantage of overestimating acidity, because its small molecular size enables access to acidic sites on the interior of the solid acid that are not accessible to typical organic substrates being employed for chemical reactions (i.e. ammonia may fit into pores that a cannabinoid may not). Despite this disadvantage, TPD with ammonia is still considered a useful technique to compare total acidity of heterogeneous solid acids (larger NH₃ absorption values correlate with stronger acidity).

Another commonly used method for characterizing the acidity of heterogeneous solid acids is microcalorimetry. In this technique, the heat of adsorption is measured when acidic sites on the solid acid are neutralized by addition of a base. The measured heat of adsorption is used to characterize the strength of Brønsted-acid sites (the larger the heat of adsorption, the stronger the acidic site, such that more negative values correlate with stronger acidity).

Microcalorimetry may provide the advantage of being a more direct method for the determination of acid strength when compared to TPD. However, the nature of the acidic sites cannot be determined by calorimetry alone, because adsorption may occur at Brønsted sites, Lewis sites, or a combination thereof. Further, experimentally determined heats of adsorption may be inconsistent in the literature for a given heterogeneous acid. For example, ΔH_{0ads} NH₃ values between about 100 kJ/mol and about 200 kJ/mol have been reported for H-ZSM-5. Thus, heats of adsorption determined by microcalorimetry may be best interpreted in combination with other acidity characterization methods such as TPD to properly characterize the acidity of solid heterogeneous acids.

Non-limiting examples of: (i) Hammett acidity values; (ii) TPD values with reference to ammonia; and (iii) microcalorimetry values with reference to ammonia, for a selection of Lewis-acidic heterogeneous reagents in accordance with the present disclosure are set out in TABLE 1.

TABLE 1
Non-limiting examples of: (i) Hammett acidity values; (ii) TPD values with reference
to ammonia; and (iii) microcalorimetry values with reference to ammonia.

| | | Hammett | | ΔH⁰_{ads} |
|---|---|---|---|---|
| | | Value | TPD NH₃ | NH₃ |
| Acid Reagent | Classification | (H₀) | (mmol/g) | (kJ/mol) |
| Amberlyst-35 | Ion-exchange resin | −5.6 | 5.2 | −117 |
| Amberlyst-15 | Ion-exchange resin | −4.6 | 4.6 | −116 |
| H-ZSM-5 | Microporous | −5.6 < H₀ < −3.0 | 1.0 | −145 |
| | aluminosilicate | | | |
| | (zeolite) | | | |
| H-Beta | Microporous | — | 0.65 | −120 |
| | aluminosilicate | | | |
| | (zeolite) | | | |
| Al-MCM-41 | Mesoporous | — | 0.26 | — |
| | aluminosilicate | | | |
| Montmorillonite | Phyllosilicate (clay) | −1.5 < H₀ < +3.2 | 0.18 | — |
| (K30) | | | | |

In the context of the present disclosure, a Lewis-acidic heterogeneous reagent has an acidity metric that surpasses a threshold acidity metric for the first cannabinoid when contact between the Lewis-acidic heterogeneous reagent and the first cannabinoid under reaction conditions defined by a contact temperature and a contact time converts at least a portion of the first cannabinoid into a second cannabinoid. Hammett acidity values (Hₒ), temperature-programmed desorption (TPD) values, microcalorimetry values, and combinations thereof are non-limiting ways to characterize such acidity metrics and/or threshold acidity metrics.

In select embodiments of the present disclosure, the Lewis-acidic heterogeneous reagent may have a Hammett-acidity value (Hₒ) of between about −8.0 and about 0.0. For example, the Lewis-acidic heterogeneous reagent may have a Hammett-acidity value (Hₒ) of between: (i) about −8.0 and about −7.0; (ii) about −7.0 and about −6.0; (iii) about −6.0 and about −5.0; (iv) about −5.0 and about −4.0; (v) about −4.0 and about −3.0; (vi) about −3.0 and about −2.0; (vii) about −2.0 and about −1.0; or (viii) about −1.0 and about 0.

In select embodiments of the present disclosure, the Lewis-acidic heterogeneous reagent may have a temperature-programmed desorption value of between about 7.5 and about 0.0 as determined with reference to ammonia (TPD_{NH3}). For example, the Lewis-acidic heterogeneous reagent may have a temperature-programmed desorption value of between: (i) about 7.5 and about 6.5 as determined with reference to ammonia (TPD_{NH3}); (ii) about 6.5 and about 5.5 as determined with reference to ammonia (TPD_{NH3}); (iii) about 5.5 and about 4.5 as determined with reference to ammonia (TPD_{NH3}); (iv) about 4.5 and about 3.5 as determined with reference to ammonia (TPD_{NH3}); (v) about 3.5 and about 2.5 as determined with reference to ammonia (TPD_{NH3}); (vi) about 2.5 and about 1.5 as determined with reference to ammonia (TPD_{NH3}); (vii) about 1.5 and about 0.5 as determined with reference to ammonia (TPD_{NH3}); or (viii) about 0.5 and about 0.0 as determined with reference to ammonia (TPD_{NH3}).

In select embodiments of the present disclosure, the Lewis-acidic heterogeneous reagent may have a heat of absorption value of between about −165 and about −100 as determined with reference to ammonia (ΔH^{o}_{ads NH3}). For example, the Lewis-acidic heterogeneous reagent may have a heat of absorption value of between: (i) about −165 and about −150 as determined with reference to ammonia (ΔH^{o}_{ads NH3}); (ii) about −150 and about −135 as determined with reference to ammonia (ΔH^{o}_{ads NH3}); (iii) about −135 and about −120 as determined with reference to ammonia (ΔH^{o}_{ads NH3}); (iv) about −120 and about −105 as determined with reference to ammonia (ΔH^{o}_{ads NH3}); or (v) about −105 and about −100 as determined with reference to ammonia (ΔH^{o}_{ads NH3}).

In select embodiments of the present disclosure, the Lewis-acidic heterogeneous reagent may comprise an ion-exchange resin, a microporous silicate such as a zeolite (natural or synthetic), a mesoporous silicate (natural or synthetic) and/or a phyllosilicate (such as montmorillonite).

Lewis-acidic heterogeneous reagents that comprise an ion-exchange resin may comprise acidic functional groups linked to a backbone of the polymer. Lewis-acidic heterogeneous reagents that comprise an ion-exchange resin may comprise, for example, Amberlyst polymeric resins (also commonly referred to as “Amberlite” resins). Amberlyst polymeric resins include but are not limited to Amberlyst-15, 16, 31, 33, 35, 36, 39, 46, 70, CH10, CH28, CH43, M-31, wet forms, dry forms, macroreticular forms, gel forms, H⁺ forms, Na⁺ forms, or combinations thereof). In select embodiments of the present disclosure, the Lewis-acidic heterogeneous reagent may comprise an Amberlyst resin that has a surface area of between about 20 m²/g and about 80 m²/g. In select embodiments of the present disclosure, the Lewis-acidic heterogeneous reagent may comprise an Amberlyst resin that has an average pore diameter of between about 100 Å and about 500 Å. In select embodiments of the present disclosure, the Lewis-acidic heterogeneous reagent may comprise Amberlyst-15. Amberlyst-15 is a styrene-divinylbenzene-based polymer with sulfonic acid functional groups linked to the polymer backbone. Amberlyst-15 may have the following structural formula:

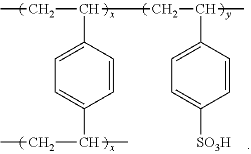 [SMILES: CCC(C)c1ccc(C(CC)CC(C)c2ccc(S(=O)(=O)O)cc2)cc1]

Lewis-acidic heterogeneous reagents that comprise an ion-exchange resin may comprise, for example, Nafion polymeric resins. Nafion polymeric resins may include but are not limited to Nafion-NR50, N115, N117, N324, N424, N1110, SAC-13, powder forms, resin forms, membrane forms, aqueous forms, dispersion forms, composite forms, H⁺ forms, Na⁺ forms, or combinations thereof.

Lewis-acidic heterogeneous reagents that comprise microporous silicates (e.g. zeolites) may comprise, for example, natural and synthetic zeolites. Lewis-acidic heterogeneous reagents that comprise mesoporous silicates may comprise, for example, Al-MCM-41 and/or MCM-41. Lewis-acidic heterogeneous reagents that comprise phyllosilicates may comprise, for example, montmorillonite. A commonality amongst these materials is that they are all silicates. Silicates may include but are not limited to Al-MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, USY, Mordenite, Ferrierite, Montmorillonite K10, Montmorillonite K20, Montmorillonite K30, KSF, Clayzic, bentonite, H⁺ forms, Na⁺ forms, or combinations thereof. Zeolites are commonly used as adsorbents and catalysts (e.g. in fluid catalytic cracking and hydrocracking in the petrochemical industry). Although zeolites are abundant in nature, the zeolites used for commercial and industrial processes are often made synthetically. Their structural framework consists of SiO₄ and AlO₄⁻ tetrahedra, which are combined in specific ratios with an amine or tetraalkylammonium salt “template” to give a zeolite with unique acidity, shape and pore size. The Lewis and/or Brønsted-Lowry acidity of zeolites can typically be modified using two approaches. One approach involves adjusting the Si/Al ratio. Since an AlO₄⁻ moiety is unstable when attached to another AlO₄⁻ unit, it is necessary for them to be separated by at least one SiO₄ unit. The strength of the individual acidic sites may increase as the AlO₄⁻ units are further separated Another approach involves cation exchange. Since zeolites contain charged AlO₄⁻ species, an extra-framework cation such as Na⁺ is required to maintain electroneutrality. The extra-framework cations can be replaced with protons to generate the “H-form” zeolite, which has stronger Brønsted acidity than its metal cation counterpart.

In select embodiments of the present disclosure, the Lewis-acidic heterogeneous reagent may comprise “H⁺-form” zeolites “Na⁺-form” zeolites, and/or a suitable mesoporous material. By way of non-limiting example, the acidic heterogeneous reagent may comprise Al-MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, USY, Mordenite, Ferrierite, Montmorillonite, Bentonite, or combinations thereof. Suitable mesoporous materials and zeolites may have a pore diameter ranging from about 0.1 nm to about 100 nm, particle sizes ranging from about 0.1 ∥m to about 50 μm, Si/Al ratio ranging from 5-1500, and any of the following cations: H⁺, Li⁺, Na⁺, K⁺, NH₄⁺, Rb⁺, Cs⁺, Ag⁺. Furthermore, suitable zeolites may have frameworks that are substituted with or coordinated to other atoms including, for example, titanium, copper, iron, cobalt, manganese, chromium, zinc, tin, zirconium, and gallium.

In select embodiments of the present disclosure, the Lewis-acidic heterogeneous reagent is H-ZSM-5 (P-38 (Si/Al=38), H⁺ form, ˜5 angstrom pore size, 2 μm particle size commercially available from ACS Materials), Na-ZSM-5 (P-38 (Si/Al=38), Na⁺ form, ˜5 angstrom pore size, 2 μm particle size commercially available from ACS Materials), Al-MCM-41 (aluminum-doped Mobil Composition of Matter No. 41; e.g., P-25 (Si/Al=25), 2.7 nm pore diameter commercially available from ACS Materials), or combinations thereof.

As will be appreciated by those skilled in the art who have benefitted from the teachings of the present disclosure, a payload composition in accordance with the present disclosure may comprise a plurality of cannabinoids (i.e. the first cannabinoid may be a mixture of cannabinoids). Accordingly, operating a vape device comprising a cartridge in accordance with the present disclosure may lead to a vapor-phase converted composition comprising a variety of cannabinoids—at least one of which was converted in contact with the acidic heterogeneous reagent.

As will be appreciated by those skilled in the art who have benefitted from the teachings of the present disclosure, converting a first cannabinoid into a second cannabinoid in the vapor-phase may lead primarily to a single cannabinoid product such as shown in EQN. 1, EQN. 2, and EQN. 3.

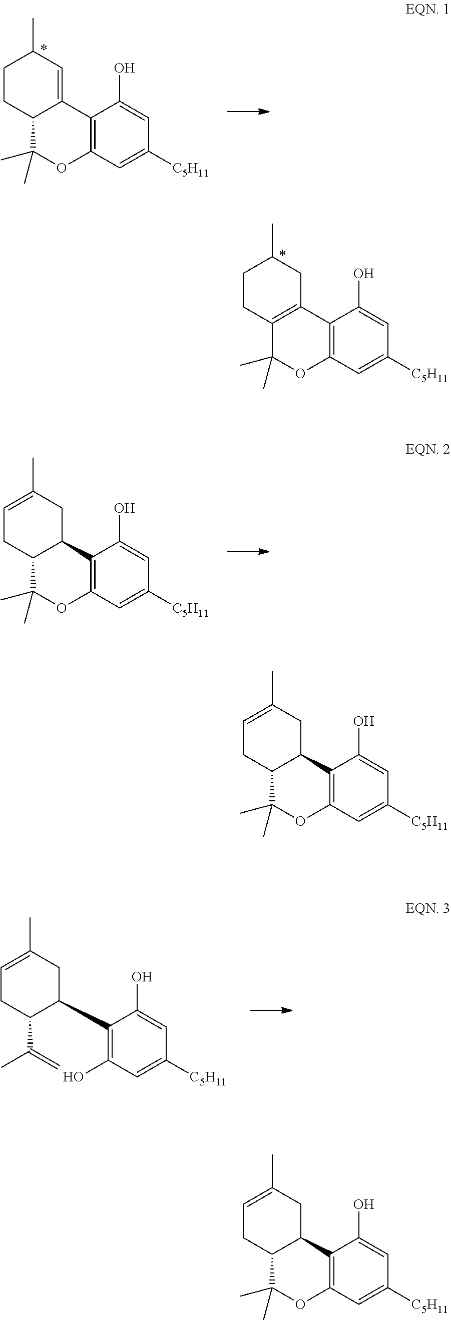

As will be appreciated by those skilled in the art who have benefitted from the teachings of the present disclosure, converting a first cannabinoid into a second cannabinoid in the vapor-phase may lead to a mixture of products such as shown in EQN. 4.

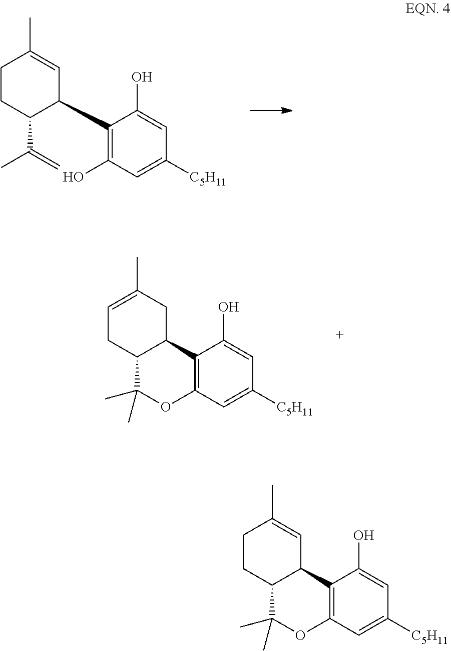

Accordingly, cartridges in accordance with the present disclosure may be paired with a variety of payload compositions and configured for use under of a variety of conditions, and these factors taken together may dictate the particular compositions provided for inhalation from the vape device.

As will be appreciated by those skilled in the art who have benefitted from the teachings of the present disclosure, a payload composition in accordance with the present disclosure may further comprise an excipient, a solvent, a diluent, an oil, a carrier fluid, and/or the like.

In select embodiments, the cartridges of the present disclosure may be configured to provide particular reaction temperatures, reagent stoichiometries, or combinations thereof. By way of non-limiting example, cartridges of the present disclosure may be configured to provide contact temperatures ranging from about 25° C. to about 300° C., for example between about 75° C. and about 100° C. The contact temperatures may be localized to the Lewis-acidic heterogeneous reagent, which may be heated by an electrical current. By way of non-limiting example, cartridges of the present disclosure may involve reagent stoichiometries ranging from about 1000:1 to about 1:1000 (cannabinoid:Lewis-acidic heterogeneous reagent—based on weight).

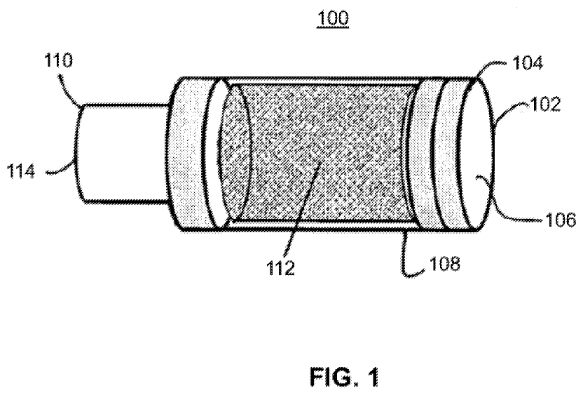

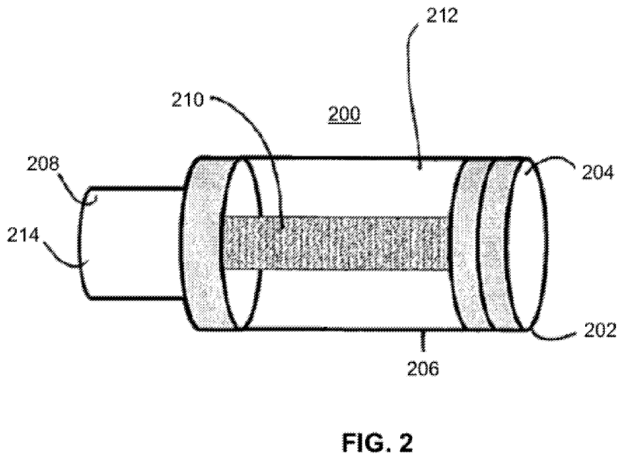

Select embodiments of the present disclosure will now be described with reference to FIG. 1 and FIG. 2, which show schematic representations of cartridges in accordance with select embodiments of the present disclosure. Cartridge 100 (FIG. 1) and cartridge 200 (FIG. 2) may each be configured to convert CBD to THC in a vapor-phase isomerization reaction. Cartridge 100 may be configured for use in connection with a vape device. Cartridge 200 may be configured for use in connection with a control assembly of a vape device. A “control assembly” as used herein may include, for example, one or more of the following: an electromechanical connector configured to engage a connector on a cartridge containing a payload reservoir and/or atomizer (commonly referred to as a “cartomizer”); a power source such as a battery; a switch or other means for causing electrical current to flow from the power source to the electromechanical connector; a microprocessor for processing instructions and controlling the power source; memory for storing instructions and data; user input device(s); display(s); and a transceiver for communicating with remote devices.

While cartridge 100 and cartridge 200 are shown as separate components that are configured for attachment to a vape device, it is within the scope of the present disclosure for cartridge 100 and/or cartridge 200 to be integrally formed with other vape device components to form a complete vape device. For example, cartridge 100 and/or cartridge 200 may be incorporated into a self-contained vape device, such as a one-piece disposable vape device or a one-piece refillable and rechargeable vape device. Further, cartridge 100 and/or 200 may be integrally formed with a payload reservoir and/or atomizer and may be configured for connection with a control assembly of a vape device.

Referring to FIG. 1, cartridge 100 may include a first end 102 with a connector 104 that is configured to connect to the mouthpiece of a vape device (not shown). The first end 102 presents an inlet 106 for receiving a vaporized payload (e.g. vaporized CBD) from the vape device. A housing 108 extends from the first end 102 to a second end 110. The housing 108 defines an interior space that is substantially filled with an acid heterogeneous reagent 112, which may be any of the Lewis-acidic heterogeneous reagents described herein (e.g., zeolite catalyst beads). Second end 110 is configured to be received in a user's mouth and includes an outlet 114 for dispensing the vaporized and converted payload (e.g. vaporized CBD converted to THC) into the user's mouth. The inlet 106 and the outlet 114 are defined by the housing 108 and are in fluid communication with the interior space containing the Lewis-acidic heterogeneous reagent 112.

In use, a user connects the connector 104 to the mouthpiece of a vape device. For example, the connector 104 may engage threads on the vape device or be pressed into frictional engagement with a portion of the vape device. Connector 104 may be a M7×0.5 mm threaded connector (commonly referred to as a “510 threaded connector”). The vape device may be any type of vape device that is configured to emit a vaporized payload (e.g. vaporized CBD). The vape device is operated by the user to vaporize the payload. The user inserts the second end 110 of cartridge 100 in their mouth and draws through outlet 114. As the user draws, the vaporized payload travels through inlet 106 into contact with the Lewis-acidic heterogeneous reagent 112. The Lewis-acidic heterogeneous reagent 112 converts the vaporized payload into the vaporized and converted payload (e.g. vaporized CBD converted to THC). The vaporized and converted payload travels through gaps in the Lewis-acidic heterogeneous reagent 112 and out the outlet 114 into the user's mouth for inhalation.

Referring to FIG. 2, cartridge 200 includes a first end 202 with a connector 204 that is configured to connect to the connector of a control assembly of a vape device (not shown). The control assembly may include, for example, an electromechanical connector configured to engage connector 204. The electromechanical connector may be a two-conductor electromechanical connector such as a 510 threaded connector.

Cartridge 200 further includes a housing 206 extending from first end 202 to a second end 208. Housing 206 defines an interior space within which is positioned an atomizer 210. The interior space includes a payload reservoir 212 that is positioned between housing 206 and atomizer 210. Payload reservoir 212 contains a payload (e.g. CBD resin). Atomizer 210 is in fluid communication with payload reservoir 212 and in contact with the payload.

Atomizer 210 is configured to heat the payload within payload reservoir 212 until the payload vaporizes. Atomizer 210 is further configured to convert the vaporized payload (e.g. vaporized CBD) into vaporized and converted payload (e.g. vaporized CBD converted to THC). Atomizer 210 may be formed as a cylindrical tube with an outer side wall and an inner side wall. Further, atomizer 210 may be formed from a porous ceramic material (e.g. a non-fibrous material such as Japanese alumina ceramic or black porous ceramic such as Al₂O₃ or black Al₂O₃) that surrounds a heating element positioned between the outer side wall and the inner side wall. The heating element may be electrically connected to connector 204 for receiving electrical current from the power source of the control assembly. The heating element may be a coil that is encased in a porous ceramic material. The heating element may be a resistive or inductive heating element and may comprise SS316L surgical stainless steel or a titanium alloy. In one embodiment, the heating element has an electrical resistance of less than 2 ohm, less than 1.5 ohm, less than 1.3 ohm or less than 1 ohm. In one embodiment, the heating element and atomizer 210 does not include nichrome or kanthal. The heating element may also be applied to the inner side wall of atomizer 210. The heating element may be configured to heat the payload up to about 200° C.

Atomizer 210 further includes a Lewis-acidic heterogeneous reagent, which may be any of the Lewis-Lewis-acidic heterogeneous reagent set out herein (e.g. zeolite). The Lewis-acidic heterogeneous reagent may be built around the heating element. For example, the Lewis-acidic heterogeneous reagent may be joined to the outer side wall or the inner side wall of the atomizer 210 in any suitable manner. Further, the Lewis-acidic heterogeneous reagent may be positioned between the outer side wall and the inner side wall, or the Lewis-acidic heterogeneous reagent may be positioned within an interior cavity defined by the inner side wall. The heating element may Lewis-acidic heterogeneous reagent along with the payload. The Lewis-acidic heterogeneous reagent is positioned so that the payload contacts the Lewis-acidic heterogeneous reagent to convert the payload while the payload vaporizes or after the payload vaporizes. For example, the catalyst is positioned to convert a payload of vaporized CBD resin into vaporized THC.

Atomizer 210 surrounds an atomizer chamber (not shown) that is in fluid communication with an outlet 214 formed in second end 208. The payload within payload reservoir 212 is in contact with the outer side wall of atomizer 210. The payload travels through the porous atomizer 210. The heating element heats and vaporizes the payload as it passes through the atomizer 210 to the atomizer chamber. The Lewis-acidic heterogeneous reagent converts the payload as it passes through the atomizer 210 to the atomizer chamber. Optionally, if the Lewis-acidic heterogeneous reagent is positioned within the atomizer chamber, the vaporized payload is converted as it travels through the atomizer chamber to the outlet 214.

In use, a user connects the connector 204 to the control assembly of a vape device. For example, the connector 204 may engage threads on the control assembly or be pressed into frictional engagement with a portion of the control assembly. Connector 204 may be a 510 threaded connector that electrically connects the heating element of atomizer 210 to a power source (e.g. a battery) of the control assembly. The control assembly is operated by the user to send electrical current from the power source of the control assembly to the heating element of the atomizer 210. The payload travels through the porous atomizer 210, is vaporized by the heating element, and then converted by the Lewis-acidic heterogeneous reagent. If the payload is CBD resin, the atomizer 210 may vaporize and convert the CBD resin into vaporized THC. The user inserts the second end 208 of cartridge 200 in the user's mouth and draws through outlet 214. As the user draws, the vaporized and converted payload is drawn into the atomizer chamber of atomizer 210, out through the outlet 214, and into the user's mouth for inhalation.

### EXAMPLES

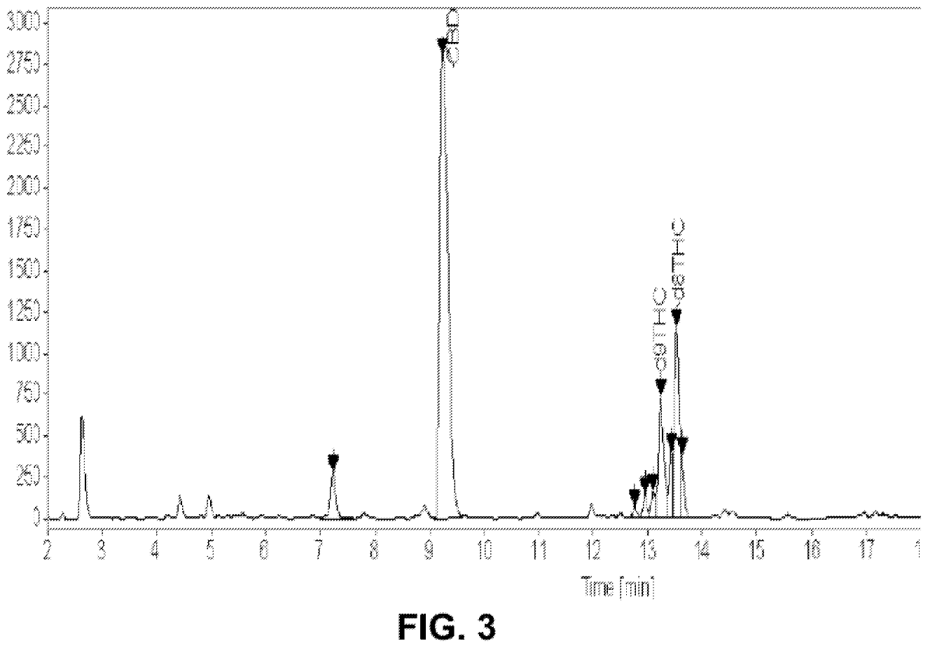

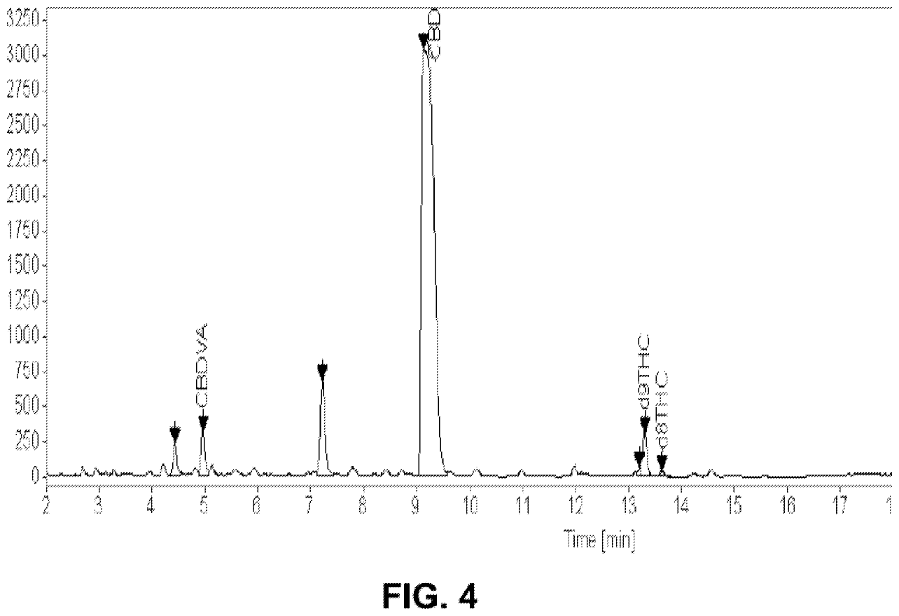

EXAMPLE 1: a Lewis-acidic heterogeneous reagent (ZSM-5, 1 g, ACS Material, P-38, H+) and a payload comprising a first cannabinoid (CBD, 500 mg, 1.59 mmol) were heated independently to greater than about 250° C. A pressure differential was created to draw vapours from the payload through a housing comprising the Lewis-acidic heterogeneous reagent. Vapours that had passed through the Lewis-acidic heterogeneous reagent were captured in a trap comprising an extraction solution. The extraction solution was analysis by HPLC (DAD 215 nm, see the chromatogram in FIG. 3). The chromatogram in FIG. 3 shows an increase in Δ⁸-THC and Δ⁹-THC as compared to the chromatograph in FIG. 4, which was collected from a control experiment as, set out in COMPARISON EXAMPLE 1. In particular, the chromatogram in FIG. 3 shows a CBD:Δ⁹-THC:Δ⁸-THC ratio of 60.33:10.41:13.86.

COMPARISON EXAMPLE 1: a payload as set out in EXAMPLE 1 was heated to greater than about 250° C. A pressure differential was created to draw vapours from the payload through a housing that was void of Lewis-acidic heterogeneous reagent (i.e. a blank housing). Vapours that had passed through the blank housing were captured in a trap comprising an extraction solution. The extraction solution was analysis by HPLC (DAD 215 nm, see the chromatogram in FIG. 4). The chromatogram in FIG. 4 shows a decrease in Δ⁸-THC and Δ⁹-THC as compared to the chromatograph in FIG. 3 which was collected from the experiment set out in EXAMPLE 1. In particular, the chromatogram in FIG. 4 shows a CBD:Δ⁹-THC:Δ⁸-THC ratio of 83.33:3.26:0.34.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1.-34. (canceled)

35. A cartridge for a vape device, the cartridge comprising:
a payload reservoir configured to contain a payload comprising a first cannabinoid to be vaporized,
an atomizer in fluid communication with the payload reservoir and configured to vaporize at least a portion of the payload to thereby generate a vaporized payload,
a Lewis-acidic heterogeneous reagent in fluid communication with the vaporized payload such that contact between the Lewis-acidic heterogeneous reagent and the vaporized payload converts at least a portion of the first cannabinoid into a second cannabinoid.

36. The cartridge of claim 35, wherein the first cannabinoid comprises cannabidiol and wherein the second cannabinoid comprises a mixture of Δ⁸-tetrahydrocannabinol and Δ⁹-tetrahydrocannabinol.

37. The cartridge of claim 35, wherein the Lewis-acidic heterogeneous reagent comprises an ion-exchange resin, a microporous silicate, a mesoporous silicate, a phyllosilicate, or any combination thereof.

38. The cartridge of claim 37, wherein the ion-exchange resin is an Amberlyst polymeric resin.

39. The cartridge of claim 38, wherein the Amberlyst polymeric resin is Amberlyst 15.

40. The cartridge of claim 37, wherein the ion-exchange resin is a Nafion polymeric resin.

41. The cartridge of claim 40, wherein the Nafion polymeric resin is Nafion NR50, N115, N117, N324, N424, N1110, SAC-13, or any combination thereof.

42. The cartridge of claim 37, wherein the microporous silicate is ZSM-5, ZSM-11, ZSM-22, ZSM-23, ZSM-35, SAPO-11, SAPO-34, SSZ-13, TS-1, Beta, X-type, Y-type, Linde type A, Linde type L, Linde type X, Linde type Y, or any combination thereof.

43. The cartridge of claim 37, wherein the mesoporous silicate is Al-MCM-41, MCM-41, MCM-48, SBA-15, SBA-16, KIT-5, KIT-6, FDU-12, or any combination thereof.

44. The cartridge of claim 37, wherein the phyllosilicate is Faujasite, Mordenite, Ferrierite, Montmorillonite K10, Montmorillonite K20, Montmorillonite K30, Montmorillonite KSF, Clayzic, bentonite, or any combination thereof.

45. The cartridge of claim 35, wherein the first cannabinoid is an acidic-form cannabinoid, and wherein the second cannabinoid is a neutral form.

46. A method for converting a first cannabinoid into a second cannabinoid, the method comprising:
vaporizing the first cannabinoid to thereby generate a vaporized first cannabinoid;
contacting the vaporized first cannabinoid with a Lewis-acidic heterogeneous reagent thereby converting at least a portion of the first cannabinoid into a second cannabinoid.

47. The method of claim 46, wherein the first cannabinoid comprises cannabidiol and wherein the second cannabinoid comprises a mixture of Δ⁸-tetrahydrocannabinol and Δ⁹-tetrahydrocannabinol.

48. The method of claim 46, wherein the Lewis-acidic heterogeneous reagent comprises an ion-exchange resin, a microporous silicate, a mesoporous silicate, a phyllosilicate, or any combination thereof.

49. The method of claim 48, wherein the ion-exchange resin is an Amberlyst polymeric resin.

50. The method of claim 49, wherein the Amberlyst polymeric resin is Amberlyst 15.

51. The method of claim 48, wherein the ion-exchange resin is a Nafion polymeric resin.

52. The method of claim 51, wherein the Nafion polymeric resin is Nafion NR50, N115, N117, N324, N424, N1110, SAC-13, or any combination thereof.

53. The method of claim 48, wherein the microporous silicate is ZSM-5, ZSM-11, ZSM-22, ZSM-23, ZSM-35, SAPO-11, SAPO-34, SSZ-13, TS-1, Beta, X-type, Y-type, Linde type A, Linde type L, Linde type X, Linde type Y, or any combination thereof.

54. The method of claim 48, wherein the mesoporous silicate is Al-MCM-41, MCM-41, MCM-48, SBA-15, SBA-16, KIT-5, KIT-6, FDU-12, or any combination thereof.

55. The method of claim 48, wherein the phyllosilicate is Faujasite, Mordenite, Ferrierite, Montmorillonite K10, Montmorillonite K20, Montmorillonite K30, Montmorillonite KSF, Clayzic, bentonite, or any combination thereof.

56. The method of claim 46, wherein the first cannabinoid is an acidic-form cannabinoid, and wherein the second cannabinoid is a neutral form.
